(19) United States
(12) Patent Application Publication
(10) Pub. No.: US 2011/0237679 A1
(43) Pub. Date: Sep. 29, 2011

(54) NOVEL ANTIVIRAL COMPOUNDS, COMPOSITIONS, AND METHODS OF USE

(75) Inventor: Thais M. Sielecki-Dzurdz, Kennett Square, PA (US)

(73) Assignee: CYTOKINE PHARMASCIENCES, INC., King of Prussia, PA (US)

(21) Appl. No.: 13/133,485

(22) PCT Filed: Dec. 9, 2009
(86) PCT No.: PCT/US09/67324

§ 371 Date: Jun. 8, 2011

Related U.S. Application Data
(60) Provisional application No. 61/120,939, filed on Dec. 9, 2008.

(51) Int. Cl. A61K 31/155; C07C 257/18; A61P 31/18; A61P 31/20; A61P 31/22; A61P 31/16

(52) U.S. Cl. 514/637; 564/245

(57) ABSTRACT

Compounds, salts thereof, and tautomers thereof are disclosed. Compositions that include the compounds are disclosed. Methods of making and using the compounds are also disclosed.

### CROSS-REFERENCE TO RELATED APPLICATIONS

This application is based on and claims priority to U.S. Provisional Application No. 61/120,939, filed Dec. 9, 2008, the entire contents of which are hereby incorporated by reference.

### BACKGROUND

1. Field

The present invention is directed to compounds, compositions, methods of making, and methods of using. Such compounds may be suitable, for example, in medicine.

2. Related Art

The relevant contents of each of the following documents are incorporated by reference: U.S. Pat. Nos. 5,574,040; 5,840,893; 5,808,068; 5,849,793; 5,877,282; and 6,297,253; U.S. Published Application No. 2005/0203150; Gendelman et al., J. Virol. 58:67-74, 1986; Gartner et al., Science 233:215-219, 1986; Langhoff et al., Proc. Natl. Acad. Sci. USA 88:998-8002, 1991; Ramazzotti et al., Immunology 85:94-98, 1995; Bukrinsky et al., Proc. Natl. Acad. Sci. USA 89:6580-6584, 1992; Bukrinsky et al., Nature 365:666-669, 1993; von Schwedler et al., Proc. Natl. Acad. Sci. USA 91:6992-6996, 1994; Lewis et al., EMBO J. 11:3053-3058, 1992; Roe et al., EMBO J. 12:2099-2108, 1993; Lewis and Emerman, J. Virol. 68:510-516, 1994; Gallay et al., J. Virol. 70:1027-1032, 1996; Popov et al., Proc. Natl. Acad. Sci. USA 93:11859-11864, 1996; Adam, Trends Cell Biol. 5:189-191, 1995; Radu et al., Proc. Natl. Acad. Sci. USA 92:1769-1773, 1995; Moroianu et al., Proc. Natl. Acad. Sci USA 92:2008-2011, 1995; Gorlich et al., Nature (London) 377:246-248, 1995; Adam and Gerace, Cell 66:837-847, 1991; Gorlich and Mattaj, Science 271:1513-1518, 1996; Hurt, Cell 84:509-515, 1996; Bukrinsky et al., Nature 365:666-669, 1993; von Schwedler et al., Proc. Natl. Acad. Sci. USA 91:6992-6996, 1994; Gallay et al., J. Virol. 70:1027-1032, 1996; Bukrinsky et al., Proc. Natl. Acad. Sci. USA 90:6125-6129, 1993; Haffar et al., J. Mol. Biol. 299: 359-368, 2000; von Schwedler et al., Proc. Natl. Acad. Sci. USA 91:6992-6996, 1994; Heizinger et al., Proc. Natl. Acad. Sci. USA 91:7311-7315, 1992; Bukrinsky et al., Nature 365:666-669, 1993; Emerman et al., Nature (London) 369:107-108, 1994; Popov et al., Proc. Natl. Acad. Sci. USA 93:11859-11864, 1996; Nadler et al., J. Biol. Chem. 272, 4310-4315, 1997; Lanford et al., Cell 15: 575-582, 1986; Lanford et al., Mol. Cell Biol. 8:2722-2729, 1988; Dworetzky et al., J. Cell Biol. 106: 575-584, 1988; Adam et al., Cell 66:837-847, 1991; Hariton-Gazal et al., Bio. Biophysic. Acta 1584: 234-242, 2002; Fletcher et al., EMBO J. 1996 Nov. 15; 15(22):6155-65; Igarashi T, et al., Proc Natl Acad Sci USA. 2001 Jan. 16; 98(2):658-63; Glushakova S, et al., Antiviral Res. 2000 August; 47(2):89-95; and Haffar O. K., et al., Antimicrob Agents Chemother. 1998 May; 42(5):1133-8.

### DESCRIPTION OF THE FIGURES

FIG. 1 provides a comparison of activity of ITI-367, ITI-367 treated with HCl for 2 hours (367 HCl) to induce ring opening, and the ring opened oxime, CPSI-00220.

FIG. 2 shows graphically the results of an in vitro toxicity evaluation of CPSI-00220, the ring opened oxime.

### DESCRIPTION OF THE SEVERAL EMBODIMENTS

One embodiment relates to a compound having one of the following formulas:

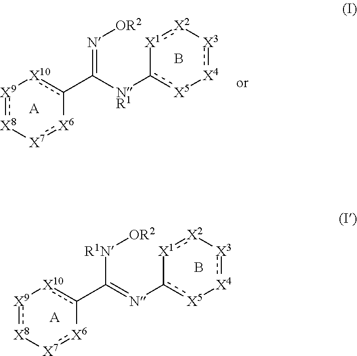

or salt or tautomer thereof.

The compounds are useful to treat and/or prevent known or suspected maladies in an animal subject. For example, the compounds may be suitably administered to a animal known or suspected to have one or more maladies such as HIV infection, hepatitis C, hepatitis B, hepatitis delta, influenza, herpes, adenovirus, papillomavirus, parvovirus, bird flu, and/or measles, which includes administering one or more of the compounds or compositions herein to the animal. Without wishing to be bound by theory, in one embodiment, these compounds may exercise their beneficial properties via a nuclear importation inhibition mechanism. In another embodiment, these compounds may exercise their beneficial properties through other mechanisms of action, including those that do not involve nuclear importation inhibition.

The animal subject may include, for example, a mammal or avian subject. Exemplary mammals include, for example, a human, dog, cat, pig, horse, cow, mouse, or the like. Exemplary avian subjects include, for example, a duck, chicken, blackbird, goose, crow, pigeon, turkey, squab, swan, and the like.

In one embodiment, the compounds are useful to treat and/or prevent known or suspected maladies in a human subject. For example, the compounds may be suitably administered to a human known or suspected to have one or more maladies such as HIV infection, hepatitis C, hepatitis B, hepatitis delta, influenza, herpes, adenovirus, papillomavirus, parvovirus, bird flu, and/or measles, which includes administering one or more of the compounds or compositions herein to the human. Without wishing to be bound by theory, these compounds may exercise their beneficial properties via a nuclear importation inhibition mechanism.

In one embodiment, the compounds may be suitably administered to a human, dog, cat, pig, horse, cow, mouse, duck, chicken, blackbird, goose, crow, pigeon, turkey, squab, swan, and the like known or suspected to have bird flu for the treatment and/or the prevention of same.

The compounds and compositions herein are useful for treating a subject suspected to have or known to have one or more of the maladies listed herein or elsewhere. As used herein, the terms “treat”, “treating” and/or “treatment” refers to acting upon with the compound or composition to improve or alter an outcome. The skilled artisan is aware that the improvement or alteration may be in whole or in part and may not be a complete cure. Treating may also including treating a subject at risk for developing one or more of the maladies recited herein or elsewhere.

In one embodiment, the compounds under proviso Rules 1-8 as described herein are excluded in accordance with the provisos.

In one embodiment, for example in a composition or method, the compounds under proviso Rules 1-8 as described herein are not excluded, i.e., the provisos do not apply.

In the formulas (I) and (I′), each [symbol] is independently a single bond or a double bond. When, in one or both of rings A and B, all of [symbol] are double bonds, the respective A or B ring may form a resonance structure.

X¹, X², X³, X⁴, X⁵, X⁶, X⁷, X⁸, X⁹, and X¹⁰ may be each independently selected from the group including —CR³═, —CR³R⁴—, —NR⁴—, —N═, and —O—.

R¹ may be selected from the group including hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof.

R² may be selected from the group including hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof.

Each of R³ and R⁴ may be independently a hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl. In one embodiment, when R³ and R⁴ are taken together on a single X, they may form an ═O group or a ═CR³R⁴ group. In one embodiment, when R³ and R⁴ are taken together with adjacent Xs, they may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof.

Each of R⁵ and R⁶ may be independently selected from the group including hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof.

Any of the (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted. If substituted, there may be one or more than one substituent. Exemplary substituents include hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof.

If either of the A or B rings are substituted, the substituent or substituents may independently appear at one or more of the ortho, para or meta positions on the respective ring. Both A and B rings may be substituted, or only the A ring, or only the B ring. Combinations of substituents and multiple substitutions are possible.

The (C₁-C₂₀)alkyl may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. It may be substituted or unsubstituted. It may be branched or unbranched.

The phenyl includes a C₆ phenyl. It may be substituted or unsubstituted.

The (C₃-C₂₀)cycloalkyl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ cycloalkyl. It may be substituted or unsubstituted. It may be branched or unbranched.

The (C₁-C₂₀)alkoxy may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkoxy. It may be substituted or unsubstituted. It may be branched or unbranched.

The (C₃-C₂₅)heteroaryl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ heteroaryl, wherein one or more of the carbons are replaced with an N, O, or S atom. More than one carbon may be replaced. Combinations of N, O, and S are possible. It may be substituted or unsubstituted. It may be branched or unbranched.

The (C₃-C₂₅)heterocyclyl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅ heterocyclyl, wherein one or more of the carbons are replaced with an N, O, or S atom. More than one carbon may be replaced. Combinations of N, O, and S are possible. It may be substituted or unsubstituted. It may be branched or unbranched.

The (C₂-C₂₀)alkenyl may be a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkenyl. It may be substituted or unsubstituted. It may be branched or unbranched.

The (C₄-C₂₀)cycloalkenyl may be a C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ cycloalkenyl. It may be substituted or unsubstituted. It may be branched or unbranched.

The (C₂-C₂₀)alkynyl may be a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkynyl. It may be substituted or unsubstituted. It may be branched or unbranched.

The (C₆-C₂₀)cycloalkynyl may be a C₆, C₇, C₈, C₉, C₁₀, C₁₁, C₁₂, C₁₃, C₁₄, C₁₅, C₁₆, C₁₇, C₁₈, C₁₉, or C₂₀ cycloalkynyl. It may be substituted or unsubstituted. It may be branched or unbranched.

The (C₅-C₂₅)aryl may be a C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ aryl. It may be substituted or unsubstituted. It may be branched or unbranched.

The perhalo(C₁-C₂₀)alkyl may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl in which all or some of the hydrogens are replaced by halogens, F, Cl, Br, or I. Combinations of halogens are possible. It may be substituted or unsubstituted. It may be branched or unbranched.

The (C₃-C₆)cycloalkyl structure may be a C₃, C₄, C₅, or C₆ cycloalkyl structure, formed from R³ and R⁴ groups taken together on adjacent Xs. The cycloalkyl structure may be substituted or unsubstituted. It may be branched or unbranched.

The (C₅-C₆)aryl structure may be a C₅ or C₆ aryl structure, formed from R³ and R⁴ groups taken together on adjacent Xs. The cycloalkyl structure may be substituted or unsubstituted. It may be branched or unbranched.

The (C₃-C₆)heteroaryl structure may be a C₃, C₄, C₅, or C₆ heteroaryl structure, formed from R³ and R⁴ groups taken together on adjacent Xs, wherein one or more of the carbons are replaced with an N, O, or S atom. More than one carbon may be replaced. Combinations of N, O, and S are possible. The cycloalkyl structure may be substituted or unsubstituted. It may be branched or unbranched.

The (C₃-C₆)heterocyclyl structure may be a C₃, C₄, C₅, or C₆ heterocyclyl structure, formed from R³ and R⁴ groups taken together on adjacent Xs, wherein one or more of the carbons are replaced with an N, O, or S atom. More than one carbon may be replaced. Combinations of N, O, and S are possible. The heterocyclyl structure may be substituted or unsubstituted. It may be branched or unbranched.

The (C₃-C₆)heterocycloalkenyl structure may be a C₃, C₄, C₅, or C₆ heterocycloalkenyl structure, formed from R³ and R⁴ groups taken together on adjacent Xs, wherein one or more of the carbons are replaced with an N, O, or S atom. More than one carbon may be replaced. Combinations of N, O, and S are possible. The heterocycloalkenyl structure may be substituted or unsubstituted. It may be branched or unbranched.

The (C₄-C₆)cycloalkenyl structure may be a C₄, C₅, or C₆ cycloalkenyl structure, formed from R³ and R⁴ groups taken together on adjacent Xs. The cycloalkenyl structure may be substituted or unsubstituted. It may be branched or unbranched.

In one embodiment, each of X¹, X², X³, X⁴, X⁵, X⁶, X⁷, X⁸, X⁹, and X¹⁰ is independently —CR³═ or —CR³R⁴—.

In one embodiment, at least one of X¹, X², X³, X⁴, X⁵, X⁶, X⁷, X⁸, X⁹, or X¹⁰ is NR⁴—, —N═, or —O—.

In one embodiment, at least one of X¹, X², X³, X⁴, or X⁵ is —NR⁴—, —N═, or —O—, and at least one of X⁶, X⁷, X⁸, X⁹, or X¹⁰ is —NR⁴—, —N═, or —O—.

In one embodiment, the compound has the following formula:

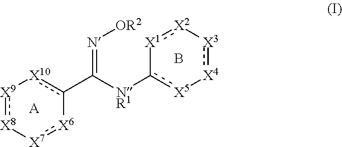

or a salt or tautomer thereof.

In one embodiment, the compound has the following formula:

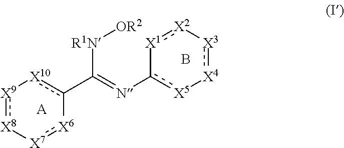

or a salt or tautomer thereof.

In one embodiment, one or both of the A and B portions of the compound having the following structures:

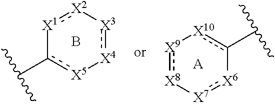

is independently a monovalent radical selected from the group including monovalent radical of any of benzene, cyclohexane, 1,2-pyran, 1,4-pyran, 1,2-pyrone, 1,4-pyrone, 1,2-dioxin, 1,3-dioxin, pyridine, pyridazine, pyrimidine, pyrazine, piperazine, 1,3,5-triazine, 1,2,4-triazine, 1,2,3-triazine, 1,2,4-oxazine, 1,3,2-oxazine, 1,3,6-oxazine, 1,2,6-oxazine, 1,4-oxazine, o-isoxazine, p-isoxazine, 1,4-oxazine, o-isoxazine, 1,4,2-oxadiazine, 1,3,5,2-oxadiazine, morpholine, indene, isoindene, benzofuran, isobenzofuran, indole, indolenine, isobenzazole, 1,5-pyrindine, pyrano[3,4-b]pyrrole, isoindazole, indoxazine, benzoxazole, anthranil, napththalene, tetralin, decalin, 1,2-benzopyran, coumarin, chromone, isocoumarin, benzopyrone, quinoline, isoquinoline, cinnoline, quinzaloline, napththryidine, pyrido[3,4-b]pyridine, pyrido[4,3-b]pyridine, 1,3,2-benzoxazine, 1,4,2-benzoxazine, 2,3,1-benzoxazine, 3,1,4-benzoxazine, 1,2-benzoxazine, 1,4-benzoxazine, purine, and a salt thereof.

In one embodiment, one or both of the A and B portions of the compound having the following structures:

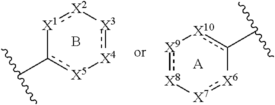

is a monovalent radical having a resonance structure.

In one embodiment, one or both of the A and B portions of the compound having the following structures:

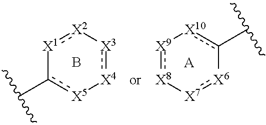

is a monovalent aromatic radical.

In one embodiment, the compound has the formula:

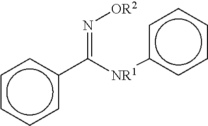

or a salt or tautomer thereof.

In one embodiment, the compound has the formula:

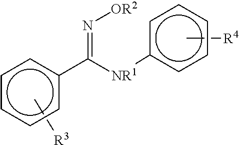

wherein one of R³ or R⁴ is not hydrogen. Salts and tautomers thereof are possible.

In one embodiment, the compound has the formula:

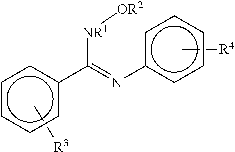

or a salt or tautomer thereof.

In one embodiment, the compound has the formula:

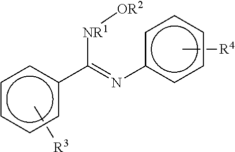

wherein one of R³ or R⁴ is not hydrogen. Salts and tautomers thereof are possible.

In one embodiment, the compound has the formula:

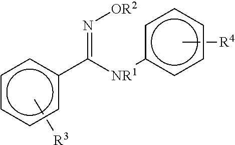

wherein R³ is OR⁵ and R⁴ is a perhalo(C₁-C₂₀)alkyl group. Salts and tautomers thereof are possible.

In one embodiment, the compound has the formula:

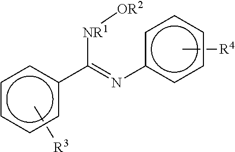

wherein R³ is OR⁵ and R⁴ is a perhalo(C₁-C₂₀)alkyl group. Salts and tautomers thereof are possible.

In one embodiment, the compound has the formula:

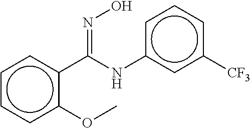

or a salt or tautomer thereof.

In one embodiment, the compound has the formula:

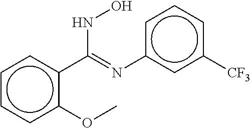

or a salt or tautomer thereof.

In one embodiment, at least one X¹, X², X³, X⁴, X⁵, X⁶, X⁷, X⁸, X⁹, or X¹⁰ is CR³R⁴— in which R³ and R⁴ are taken together to form an ═O group or a ═CR³R⁴ group.

In one embodiment, at least two adjacent X¹, X², X³, X⁴, X⁵, X⁶, X⁷, X⁸, X⁹, or X¹⁰ are each independently —CR³═, —CR³R⁴—, or —NR⁴— in which R³ and R⁴ are taken together with the adjacent Xs to form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof.

In one embodiment, the compound has one of the formulas (I) or (I′), with the proviso (rule (1)) that when N′ is ═N′— and R¹ and R² are H, and the B ring is

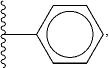

then the A ring is not:

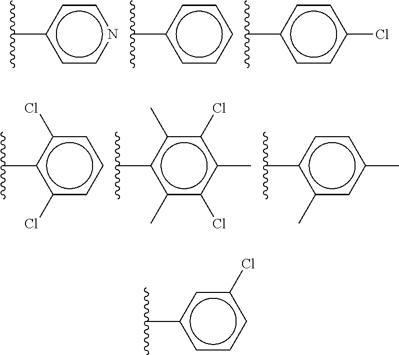

In one embodiment, the compound has one of the formulas (I) or (I′), with the proviso (Rule (2)) that when N′ is ═N′—, R² is H, R¹ is —CH₄, and the B ring is

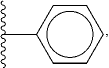

then the A ring is not:

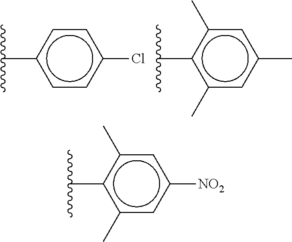

In one embodiment, the compound has one of the formulas (I) or (I′), with the proviso (Rule (3)) that when N′ is ═N′—, when R¹ and R² are H, and when the A ring is

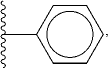

then the B ring is not

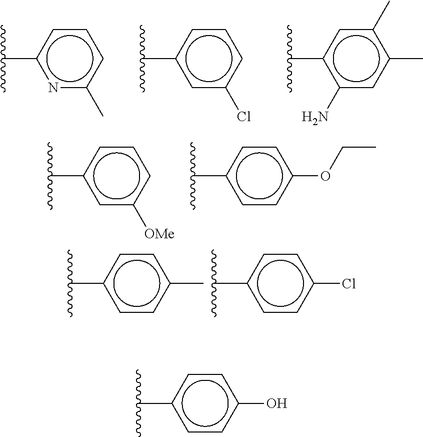

In one embodiment, the compound has one of the formulas (I) or (I′), with the proviso (Rule (4)) that when N′ is ═N′—, when R¹ and R² are both H, and the B ring is

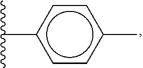

then the A ring is not:

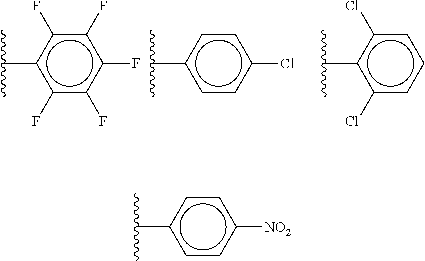

In one embodiment, the compound has one of the formulas (I) or (I′), with the proviso (Rule (5)) that when N′ is ═N′—, when R¹ and R² are both H, and the A ring is

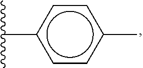

then the B ring is not

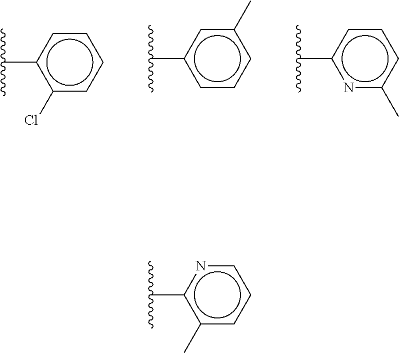

In one embodiment, the compound has one of the formulas (I) or (I′), with the proviso (Rule (6)) that when R¹ is ethyl, R² is H, when N′ is ═N′— and the A ring is

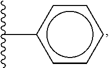

then the B ring is not

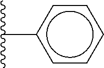

In one embodiment, the compound has one of the formulas (I) or (I′), with the proviso (Rule (7)) that when R¹ is H, R² is H, when N′ is ═N′—, and when A ring is

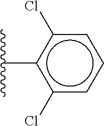

then the B ring is not

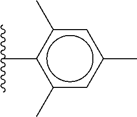

In one embodiment, the compound has one of the formulas (I) or (I′), with the proviso (Rule (8)) that when R¹ and R² are H, when N′ is ═N′—, and when B ring is

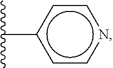

then the A ring is not

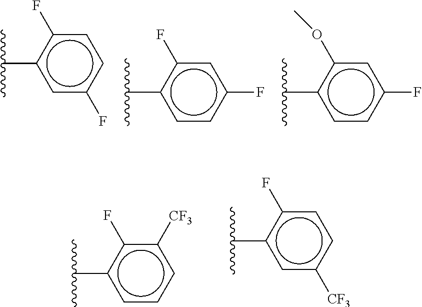

One embodiment includes a composition, in which more than one compound of formulas (I), (I′), salt thereof, or tautomer thereof, are present. For example, the composition may include a compound having formula (I) or (I′) and a salt or tautomer thereof. Another composition may include two or more different compounds of formulas (I), (I′), salt thereof or tautomer thereof. In one embodiment, the proviso Rules 1-8 do not apply to the compounds in such a composition.

The compounds having formulas (I) and (I′) may exist in the same composition as tautomers of one another. In one such embodiment, R¹ is hydrogen. Other combinations of tautomers and/or salts thereof are possible. In one embodiment, the proviso Rules 1-8 do not apply to the compounds in such a composition.

One embodiment includes a composition, in which a compound having formulas (I), (I′), salt thereof, or tautomer thereof is present, together with at least one pharmaceutically acceptable carrier. In one embodiment, the proviso Rules 1-8 do not apply to the compounds in such a composition.

One embodiment includes a composition, in which a compound having formulas (I), (I′), salt thereof, or tautomer thereof is present, together with at least one known inhibitor, such as Nucleoside analog Reverse Transcriptase inhibitor (NRTi), Non-Nucleoside analog Reverse Transcriptase inhibitor (NNRTi), Protease inhibitor (Pi), or Cell Entry inhibitor (Ci). Combinations are possible. In one embodiment, the proviso Rules 1-8 do not apply to the compounds in such a composition.

Some examples of the Nucleoside analog Reverse Transcriptase inhibitor (NRTi) include AZT, 3TC, FTC, ABC, ddC, ZDV, TDF, ddI, DFC, DAPD, alovidine, elvucitabine, D4T, RCV, or Beta-L-Fd4C. Combinations are possible.

Examples of the Non-Nucleoside analog Reverse Transcriptase inhibitor (NNRTi) include DLV, EFV, NVP, calanolide A, etravirine, TMC-278, BMS-561390, or capravirine. Combinations are possible.

Examples of the Protease inhibitor (Pi) include APV, TPV, IDV, SQV, LPV, FPV, RTV, ATZ, NFV, brecanavir, darunavir, PPL-100, L-756423, or RO033-4649. Combinations are possible.

Examples of the Cell Entry inhibitor (Ci) include Fuzeon, ENF, aplaviroc, maraviroc, vicriviroc, T-1249, PRO-542, TNX-355, SCH—C, PRO-140, SP-01A, SP-10, or TNX-355. Combinations are possible.

The inhibitors may be administered with the compound separately or in the same composition. If administered separately, the inhibitor may be administered in advance of, concurrently with, or after the administration of the compound

Given the compounds and the teachings herein, combined with the knowledge of one of ordinary skill in organic and/or synthetic chemistry, one can synthesize the compounds herein without undue effort or experimentation.

The compound may be administered to the subject in any form or mode which makes the compound bioavailable in effective amounts, or improves its bioavailability, including orally, subcutaneously, intramuscularly, intravenously, transdermally, intranasally, rectally, topically, intramucosaly, intravaginally, parenterally, and the like. Given the teachings herein, one skilled in the art can readily select the proper form and mode of administration depending upon the compound selected, the malady to be treated, the stage of the disease, and other relevant circumstances.

The compound may be administered alone or in the form of a pharmaceutical composition in combination with one or more pharmaceutically acceptable carriers so long as the composition is suitable for administration to a mammalian subject, and particularly a human. In one embodiment, one or more prodrugs of the compound are contemplated for administration. Mixtures are possible.

The compound or composition may be suitably administered batchwise or by constant or periodic infusion over an extended period of time, for example, exceeding 24 hours, until the desired therapeutic, preventive, and/or inhibiting benefits are obtained.

The carrier is physiologically tolerable by a human and does not interfere with the intended effect of the active compound. Some nonlimiting examples of the pharmaceutically acceptable carrier include water, physiological saline, ethanol, aqueous ethanol, dimethyl sulfoxide, castor oil, benzyl alcohol, benzyl benzoate, albumin, polyethylene glycol, cellulose, fatty acid, methylcellulose, dextrose, glycerol, mannitol, lactose, starch, magnesium stearate, sodium saccharin, or magnesium carbonate, or a combination thereof.

The suitability of particular carriers for inclusion in a given composition may depend on the route of administration desired. For example, the composition may be prepared as liquid solution, suspension, emulsion, cream, inhalant, patch, implant, solid, tablet, pill, capsule, sustained release, or powder form. The composition may include such one or more additives or excipients such as binder, filler, preservative, stabilizing agent, emulsifier, wetting agent, emulsifying agent, stabilizing agent, pH buffering agent, and the like. Combinations are possible.

The composition may contain 1% to 95% by weight of active compound, as appropriate, be it the compound, salt, tautomer, prodrug, or a combination thereof. This range includes all values and subranges therebetween, including 1, 2, 3, 4, 5, 6, 7, 8, 9, 10, 20, 30, 40, 50, 60, 70, 80, 90, and 95% by weight, and any combination thereof.

The term “therapeutically effective amount” refers to an amount of the compound, prodrug, salt, or combination thereof which is effective, upon single or multiple dose administration or continuous administration, infusion or application to the patient, for the treatment of the malady. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to the subjects size, age, sex, and general health; the malady involved; the degree of or involvement or the severity of the disease; the response of the individual subject; the particular compound administered; the bioavailability characteristics of the preparation administered; the dose regimen selected; the use of concomitant medication; and other relevant circumstances.

A therapeutically effective amount of the compound may range from about 0.0001 milligram per kilogram of body weight per day (mg/kg/day) to about 10,000 mg/kg/day. Preferred amounts may range from about 0.001 to about 100 mg/kg/day. These ranges include all values and subranges therebetween, including 0.0001, 0.0005, 0.001, 0.005, 0.01, 0.05, 0.1, 0.5, 1, 2, 3, 4, 5, 6, 7, 8, 9, 10, 100, 1,000, 5,000, and 10,000 mg/kg/day, and any combination thereof.

### EXAMPLES

The following examples are provided for the purpose of illustration and are not intended to be limiting.

Preparation of hydroxyiminoyl chloride: N-chlorosuccinimide (NCS) was added to a solution of o-methoxybenzaldehyde oxime in anhydrous THF (20 mL) at 0° C. under argon. The solution was stirred at the same temperature for two hours and 20 minutes. Reaction progress was monitored by TLC using hexanes/ethyl acetate (3:1).

| | Chemicals/Reagents & | | | | |
|---|---|---|---|---|---|
| S. No. | Solvents | MW | mmol | Eq. | Amts. |
| 1 | O-methoxybenzaldehyde | 151.17 | 6.91 | 1.0 | 1.045 g |
| | oxime | | | | |
| 2 | THF (tetrahydrofuran) | | | | 35 mL |
| 3 | NCS (N- | 133.53 | 10.365 | 1.5 | 1.384 g |
| | chlorosuccinimide) | | | | |

Reaction time: 2 h 20 min.
Reaction temperature: 0° C.

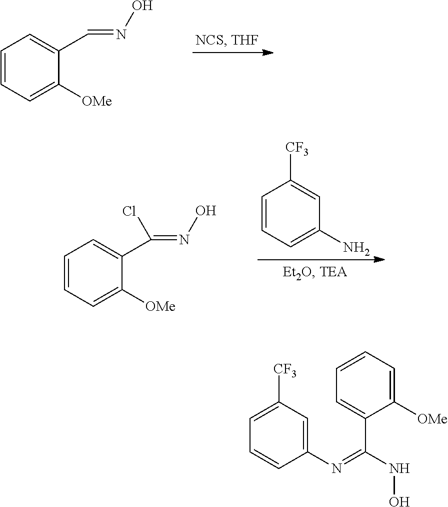

Work Up: The reaction mixture was concentrated in vacuo, and the residue was taken up in EtOAc, washed with water, brine, and dried with anhydrous sodium sulfate. The crude was concentrated in vacuo again and used directly for the next step without further purification.

### Preparation of N-hydroxy-2-methoxy-N′-(3-trifluoromethyl-phenyl)-benzamidine

| | Chemicals/Reagents & | | | | |
|---|---|---|---|---|---|
| S. No. | Solvents | MW | mmol | Eq. | Amts. |
| 1 | Hydroxyiminoyl chloride | 185 | 3.45 | 1.0 | ~0.83 g |
| | | | | | (contains |
| | | | | | some |
| | | | | | impurities) |
| 2 | Anhydrous Et₂O (ethyl | | | | 30 mL |
| | ether) | | | | |
| 3 | 3-(trifluoromethyl)- | 161.13 | 34.5 | 10 | 4.3 mL |
| | aniline | d = 1.295 | | | |
| 4 | TEA (triethyleamine) | 101 | 3.45 | 1 | 480 μL |
| | | d = 0.726 | | | |

Reaction time: 60 h.
Reaction temperature: room temperature.

Procedure: To a solution of hydroxyiminoyl chloride in anhydrous Et₂O was added 3-(trifluoromethyl)-aniline in one portion. TEA was added dropwise during 5 minutes. The resulting solution was stirred at room temperature for 60 hours under argon. Reaction progress was monitored by TLC using hexanes/ethyl acetate (3:1).

Work up: The reaction mixture was concentrated in vacuo, and the residue was directly loaded to a silicon column. Elution of the column with hexanes and ethyl acetate from 3:1 to 1.5 to 1 afforded the desired intermediate 477 mg, yield 45% for two steps. Both proton NMR and LC-MS confirmed the formation of the desired adduct intermediate.

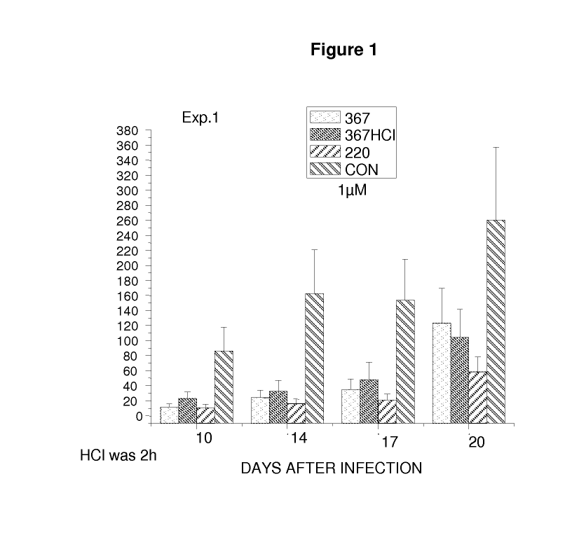

The present inventors have synthesized the ring open degradant of ITI-367 (CPSI-00220, FIG. 1) and shown that it is equally (and possibly more) as effective as ITI-367 in vitro. In FIG. 1, each set of data bars (four data bars per set) lists the results obtained for ITI-367, ITI-367HCl, CPSI-00220, and CONT, as read from left to right, which corresponds to the legend, 367, 367HCl, 220, and CONT, as read from top to bottom. ITI-367 is a known compound, which has the following formula:

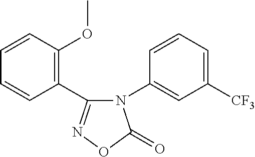

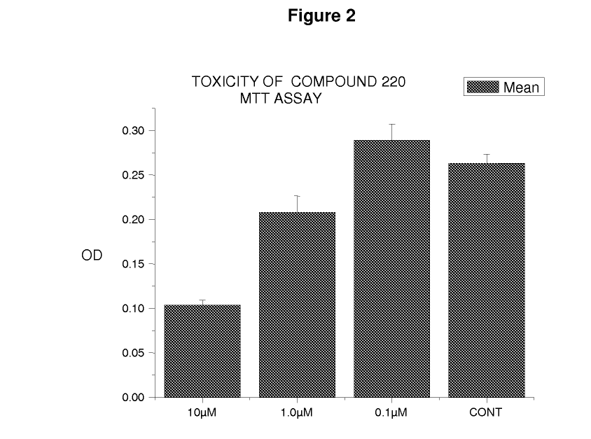

The ring open degradant is an oxime. The oxime series of compounds show some in vitro toxicity at the higher concentration of 10 uM, but are active at the non-toxic concentration of 1 μM (FIG. 2).

What is claimed is:

1. A compound having one of the following formulas: 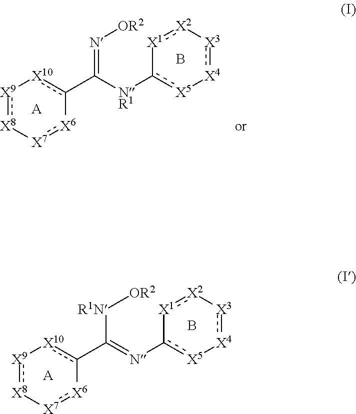
or salt thereof;
or tautomer thereof;
wherein each [symbol] is independently a single bond or a double bond;
wherein when, in one or both of rings A and B, all of [symbol] are double bonds, the respective A or B ring may form a resonance structure;
wherein X¹, X², X³, X⁴, X⁵, X⁶, X⁷, X⁸, X⁹, and X¹⁰ are each independently selected from the group consisting of —CR³═, —CR³R⁴—, —NR⁴—, —N═, and —O—;
wherein R¹ is selected from the group consisting of hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R² is selected from the group consisting of hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R³ and R⁴ are each independently hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl, or, when R³ and R⁴ are taken together on a single X, may form an ═O group or a ═CR³R⁴ group, or, when R³ and R⁴ are taken together with adjacent Xs, may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof;
wherein R⁵ and R⁶ are each independently selected from the group consisting of hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
and wherein each of said (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted by one or more substituents selected from the group consisting of hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof;
with the provisos that:
Rule (1) when N′ is αN′— and R¹ and R² are H, and the B ring is 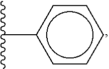
then the A ring is not: 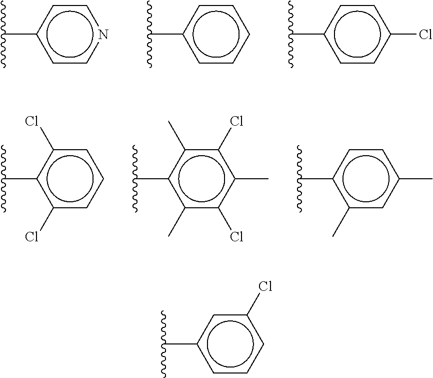
Rule (2) when N′ is ═N′—, R² is H, R¹ is —CH₄, and the B ring is 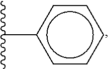
then the A ring is not: 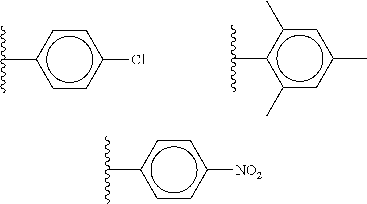
Rule (3) when N′ is ═N′—, when R¹ and R² are H, and when the A ring is 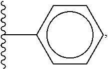
then the B ring is not 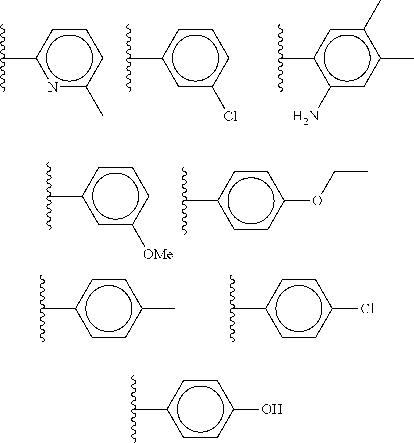
Rule (4) when N′ is ═N′—, when R¹ and R² are both H, and the B ring is 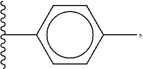
then the A ring is not: 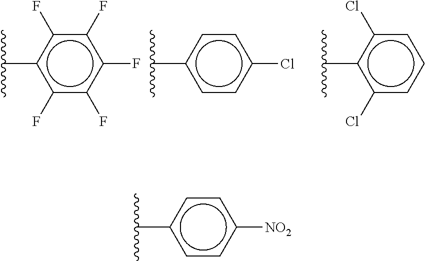
Rule (5) when N′ is ═N′—, when R¹ and R² are both H, and the A ring is 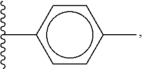
then the B ring is not 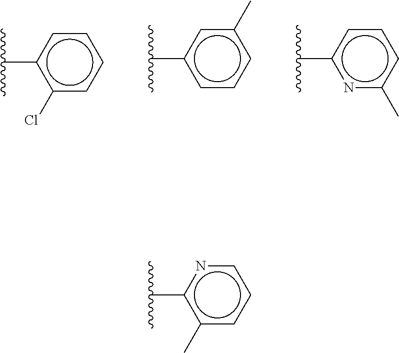
Rule (6) when R¹ is ethyl, R² is H, when N′ is ═N′— and the A ring is 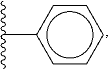
then the B ring is not 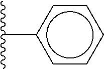
Rule (7) when R¹ is H, R² is H, when N′ is ═N′—, and when A ring is 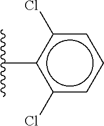
then the B ring is not 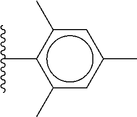
Rule (8) when R¹ and R² are H, when N′ is ═N′—, and when B ring is 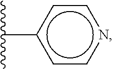
then the A ring is not

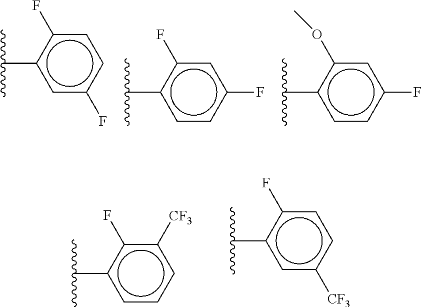

2. The compound of claim 1, wherein each of X¹, X², X³, X⁴, X⁵, X⁶, X⁷, X^{8, X}⁹, and X¹⁰ is independently —CR³═ or —CR³R⁴—.

3. The compound of claim 1, wherein at least one of X¹, X², X³, X⁴, X⁵, X⁶, X⁷, X⁸, X⁹, or X¹⁰ is —NR⁴—, —N═, or —O—.

4. The compound of claim 1, wherein at least one of X¹, X², X³, X⁴, or X⁵ is —NR⁴—, —N═, or —O—; and wherein at least one of X⁶, X⁷, X⁸, X⁹, or X¹⁰ is —NR⁴—, —N═, or —O—.

5. The compound of claim 1, which has the following formula: 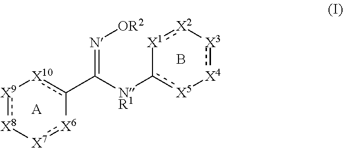
or salt thereof;
or tautomer thereof.

6. The compound of claim 1, which has the following formula: 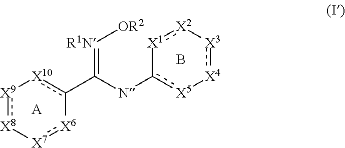
or salt thereof;
or tautomer thereof.

7. The compound of claim 1, wherein one or both of the A and B portions of the compound having the following structures: 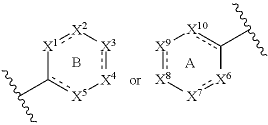
is independently a monovalent radical selected from the group consisting of monovalent radical of benzene, cyclohexane, 1,2-pyran, 1,4-pyran, 1,2-pyrone, 1,4-pyrone, 1,2-dioxin, 1,3-dioxin, pyridine, pyridazine, pyrimidine, pyrazine, piperazine, 1,3,5-triazine, 1,2,4-triazine, 1,2,3-triazine, 1,2,4-oxazine, 1,3,2-oxazine, 1,3,6-oxazine, 1,2,6-oxazine, 1,4-oxazine, o-isoxazine, p-isoxazine, 1,4-oxazine, o-isoxazine, 1,4,2-oxadiazine, 1,3,5,2-oxadiazine, morpholine, indene, isoindene, benzofuran, isobenzofuran, indole, indolenine, isobenzazole, 1,5-pyrindine, pyrano[3,4-b]pyrrole, isoindazole, indoxazine, benzoxazole, anthranil, napththalene, tetralin, decalin, 1,2-benzopyran, coumarin, chromone, isocoumarin, benzopyrone, quinoline, isoquinoline, cinnoline, quinzaloline, napththryidine, pyrido[3,4-b]pyridine, pyrido[4,3-b]pyridine, 1,3,2-benzoxazine, 1,4,2-benzoxazine, 2,3,1-benzoxazine, 3,1,4-benzoxazine, 1,2-benzoxazine, 1,4-benzoxazine, purine, and a salt thereof.

8. The compound of claim 1, wherein one or both of the A and B portions of the compound having the following structures: 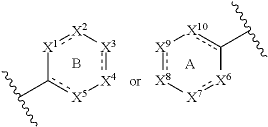
is a monovalent radical having a resonance structure.

9. The compound of claim 1, wherein one or both of the A and B portions of the compound having the following structures: 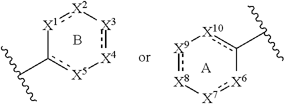
is a monovalent aromatic radical.

10. The compound of claim 1, which has the formula: 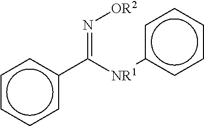
or salt thereof;
or tautomer thereof.

11. The compound of claim 1, having the formula: 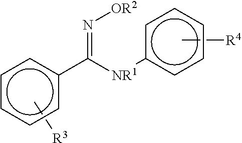
wherein one of R³ or R⁴ is not hydrogen;
or salt thereof;
or tautomer thereof.

12. The compound of claim 1, having the formula: 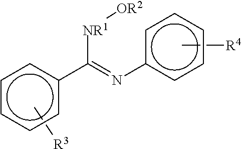
or salt thereof;
or tautomer thereof.

13. The compound of claim 1, having the formula: 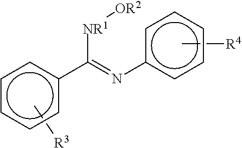
wherein one of R³ or R⁴ is not hydrogen;
or salt thereof;
or tautomer thereof.

14. The compound of claim 1, having the formula: 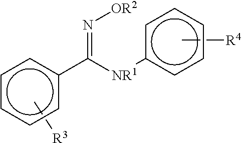
wherein R³ is OR⁵ and R⁴ is a perhalo(C₁-C₂₀)alkyl group;
or salt thereof;
or tautomer thereof.

15. The compound of claim 1, having the formula: 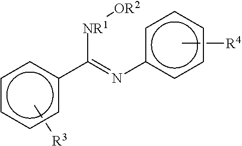
wherein R³ is OR⁵ and R⁴ is a perhalo(C₁-C₂₀)alkyl group;
or salt thereof;
or tautomer thereof.

16. The compound of claim 1, having the formula: 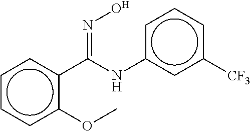
or salt thereof;
or tautomer thereof.

17. The compound of claim 1, having the formula: 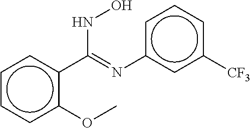
or salt thereof;
or tautomer thereof.

18. The compound of claim 1, wherein at least one X¹, X², X³, X⁴, X⁵, X⁶, X⁷, X⁸, X⁹, or X¹⁰ is —CR³R⁴— in which R³ and R⁴ are taken together to form an ═O group or a ═CR³R⁴ group.

19. The compound of claim 1, wherein at least two adjacent X¹, X², X³, X⁴, X⁵, X⁶, X⁷, X⁸, X⁹, or X¹⁰ are each independently —CR³═, —CR³R⁴—, or —NR⁴— in which R³ and R⁴ are taken together with the adjacent Xs to form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof.

20. A composition, comprising at least two different compounds having one of the following formulas: 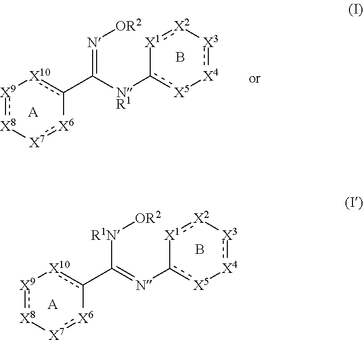
or salt thereof;
or tautomer thereof;
wherein each [symbol] is independently a single bond or a double bond;
wherein when, in one or both of rings A and B, all of [symbol] are double bonds, the respective A or B ring may form a resonance structure;
wherein X¹, X², X³, X⁴, X⁵, X⁶, X⁷, X⁸, X⁹, and X¹⁰ are each independently selected from the group consisting of —CR³═, —CR³R⁴—, —NR⁴—, —N═, and —O—;
wherein R¹ is selected from the group consisting of hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R² is selected from the group consisting of hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R³ and R⁴ are each independently hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl, or, when R³ and R⁴ are taken together on a single X, may form an ═O group or a ═CR³R⁴ group, or, when R³ and R⁴ are taken together with adjacent Xs, may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof;
wherein R⁵ and R⁶ are each independently selected from the group consisting of hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
and wherein each of said (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted by one or more substituents selected from the group consisting of hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof.

21. The composition of claim 20, wherein the composition comprises a compound having formula (I) or (I′) and a salt or tautomer thereof.

22. A composition, comprising at least one pharmaceutically acceptable carrier and at least one compound having one of the following formulas: 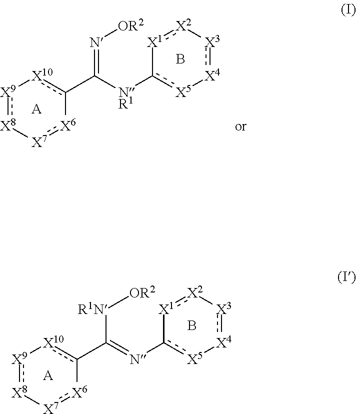
or salt thereof;
or tautomer thereof;
wherein each [symbol] is independently a single bond or a double bond;
wherein when, in one or both of rings A and B, all of [symbol] are double bonds, the respective A or B ring may form a resonance structure;
wherein X¹, X², X³, X⁴, X⁵, X⁶, X⁷, X⁸, X⁹, and X¹⁰ are each independently selected from the group consisting of —CR³═, —CR³R⁴—, —NR⁴—, —N═, and —O—;
wherein R¹ is selected from the group consisting of hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R² is selected from the group consisting of hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R³ and R⁴ are each independently hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl, or, when R³ and R⁴ are taken together on a single X, may form an ═O group or a ═CR³R⁴ group, or, when R³ and R⁴ are taken together with adjacent Xs, may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof;
wherein R⁵ and R⁶ are each independently selected from the group consisting of hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
and wherein each of said (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted by one or more substituents selected from the group consisting of hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof.

23. A composition, comprising:
at least one inhibitor selected from the group consisting of Nucleoside analog Reverse Transcriptase inhibitor (NRTi), Non-Nucleoside analog Reverse Transcriptase inhibitor (NNRTi), Protease inhibitor (Pi), Cell Entry inhibitor (Ci), and a combination thereof; and
at least one compound having one of the following formulas: 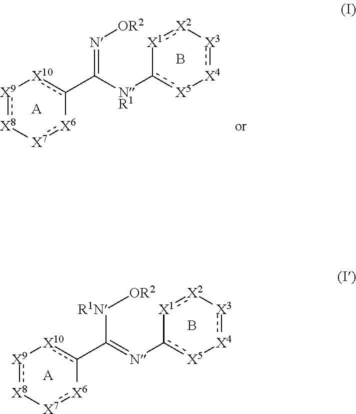
or salt thereof;
or tautomer thereof;
wherein each [symbol] is independently a single bond or a double bond;
wherein when, in one or both of rings A and B, all of [symbol] are double bonds, the respective A or B ring may form a resonance structure;
wherein X¹, X², X³, X⁴, X⁵, X⁶, X⁷, X⁸, X⁹, and X¹⁰ are each independently selected from the group consisting of —CR³═, —CR³R⁴—, —NR⁴—, —N═, and —O—;
wherein R¹ is selected from the group consisting of hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R² is selected from the group consisting of hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R³ and R⁴ are each independently hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl, or, when R³ and R⁴ are taken together on a single X, may form an ═O group or a ═CR³R⁴ group, or, when R³ and R⁴ are taken together with adjacent Xs, may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof;
wherein R⁵ and R⁶ are each independently selected from the group consisting of hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
and wherein each of said (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted by one or more substituents selected from the group consisting of hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof.

24. The composition of claim 23, wherein the inhibitor is a Nucleoside analog Reverse Transcriptase inhibitor (NRTi).

25. The composition of claim 23, wherein the inhibitor is a Nucleoside analog Reverse Transcriptase inhibitor (NRTi) selected from the group consisting of AZT, 3TC, FTC, ABC, ddC, ZDV, TDF, ddI, DFC, DAPD, alovidine, elvucitabine, D4T, RCV, Beta-L-Fd4C, and a combination thereof.

26. The composition of claim 23, wherein the inhibitor is a Non-Nucleoside analog Reverse Transcriptase inhibitor (NNRTi).

27. The composition of claim 23, wherein the inhibitor is a Non-Nucleoside analog Reverse Transcriptase inhibitor (NNRTi) selected from the group consisting of DLV, EFV, NVP, calanolide A, etravirine, TMC-278, BMS-561390, capravirine, and a combination thereof.

28. The composition of claim 23, wherein the inhibitor is a Protease inhibitor (Pi).

29. The composition of claim 23, wherein the inhibitor is a Protease inhibitor (Pi) selected from the group consisting of APV, TPV, IDV, SQV, LPV, FPV, RTV, ATZ, NFV, brecanavir, darunavir, PPL-100, L-756423, RO033-4649, and a combination thereof.

30. The composition of claim 23, wherein the inhibitor is a Cell Entry inhibitor (Ci).

31. The composition of claim 23, wherein the inhibitor is a Cell Entry inhibitor (Ci) selected from the group consisting of Fuzeon, ENF, aplaviroc, maraviroc, vicriviroc, T-1249, PRO-542, TNX-355, SCH—C, PRO-140, SP-01A, SP-10, TNX-355, and a combination thereof.

32. The composition of claim 23, further comprising a pharmaceutically acceptable carrier.

33. A method for treating a known or suspected malady, in an animal, selected from the group consisting of HIV infection, hepatitis C, hepatitis B, hepatitis delta, influenza, herpes, adenovirus, papillomavirus, parvovirus, measles, bird flu, and a combination thereof, comprising administering, to the animal, at least one compound having one of the following formulas: 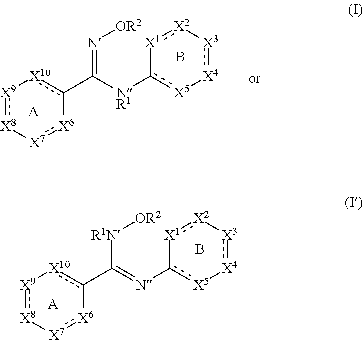
or salt thereof;
or tautomer thereof;
wherein each [symbol] is independently a single bond or a double bond;
wherein when, in one or both of rings A and B, all of [symbol] are double bonds, the respective A or B ring may form a resonance structure;
wherein X¹, X², X³, X⁴, X⁵, X⁶, X⁷, X⁸, X⁹, and X¹⁰ are each independently selected from the group consisting of —CR³═, —CR³R⁴—, —NR⁴—, —N═, and —O—;
wherein R¹ is selected from the group consisting of hydrogen, hydroxyl, —OR⁵, —NR⁵R⁶, —C(NR⁵)R⁶, —C(NR⁵)OR⁶, —NO₂, —NH₂, —CONR⁵R⁶, —COR⁵, —COOR⁵, —SO₂R⁵, —C(COR⁵)R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R² is selected from the group consisting of hydrogen, —R⁵OR⁶, —COOR⁵, —COR⁵, —PO₃R⁵R⁶, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
wherein R³ and R⁴ are each independently hydrogen, hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, or perhalo(C₁-C₂₀)alkyl, or, when R³ and R⁴ are taken together on a single X, may form an ═O group or a ═CR³R⁴ group, or, when R³ and R⁴ are taken together with adjacent Xs, may form a (C₃-C₆)cycloalkyl structure, a (C₅-C₆)aryl structure, a (C₃-C₆)heteroaryl structure, a (C₃-C₆)heterocyclyl structure, a (C₃-C₆)heterocycloalkenyl structure, or a (C₄-C₆)cycloalkenyl structure, or salt thereof;
wherein R⁵ and R⁶ are each independently selected from the group consisting of hydrogen, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, and salt thereof;
and wherein each of said (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, (C₃-C₆)cycloalkyl structure, (C₅-C₆)aryl structure, (C₃-C₆)heteroaryl structure, (C₃-C₆)heterocyclyl structure, a (C₃-C₆)heterocycloalkenyl structure, or (C₄-C₆)cycloalkenyl structure may be independently unsubstituted or substituted by one or more substituents selected from the group consisting of hydroxyl, halo, bromo, chloro, iodo, fluoro, —OR⁵, —NR⁵R⁶, —NR⁵COR⁶, —CONR⁵R⁶, —CONR⁵, —COOR⁵, —OCOR⁵, —COR⁵, —SR⁵, —SO₂R⁵, —SO₃R⁵, —SO₂NR⁵, —SOR⁵, —N₃, —CN, —NC, —SH, —NO₂, —NH₂, —PR₂, —(O)PR⁵R⁶, —PO₃R⁵R⁶, —OPO₃R⁵R⁶, —PO₂, (C₁-C₂₀)alkyl, phenyl, (C₃-C₂₀)cycloalkyl, (C₁-C₂₀)alkoxy, (C₃-C₂₅)heteroaryl, (C₃-C₂₅)heterocyclyl, (C₂-C₂₀)alkenyl, (C₄-C₂₀)cycloalkenyl, (C₂-C₂₀)alkynyl, (C₆-C₂₀)cycloalkynyl, (C₅-C₂₅)aryl, perhalo(C₁-C₂₀)alkyl, salt thereof, and a combination thereof.

34. The method of claim 33, further comprising administering at least one inhibitor selected from the group consisting of Nucleoside analog Reverse Transcriptase inhibitor (NRTi), Non-Nucleoside analog Reverse Transcriptase inhibitor (NNRTi), Protease inhibitor (Pi), Cell Entry inhibitor (Ci), and a combination thereof.

35. The method of claim 33, wherein the inhibitor is a Nucleoside analog Reverse Transcriptase inhibitor (NRTi) selected from the group consisting of AZT, 3TC, FTC, ABC, ddC, ZDV, TDF, ddI, DFC, DAPD, alovidine, elvucitabine, and a combination thereof.

36. The method of claim 33, wherein the inhibitor is a Non-Nucleoside analog Reverse Transcriptase inhibitor (NNRTi).

37. The method of claim 33, wherein the inhibitor is a Non-Nucleoside analog Reverse Transcriptase inhibitor (NNRTi) selected from the group consisting of DLV, EFV, NVP, calanolide A, etravirine, TMC-278, BMS-561390, capravirine, and a combination thereof.

38. The method of claim 33, wherein the inhibitor is a Protease inhibitor (Pi).

39. The method of claim 33, wherein the inhibitor is a Protease inhibitor (Pi) selected from the group consisting of APV, TPV, IDV, SQV, LPV, FPV, RTV, ATZ, NFV, brecanavir, darunavir, and a combination thereof.

40. The method of claim 33, wherein the inhibitor is a Cell Entry inhibitor (Ci).

41. The method of claim 33, wherein the inhibitor is a Cell Entry inhibitor (Ci) selected from the group consisting of Fuzeon, ENF, aplaviroc, maraviroc, vicriviroc, T-1249, PRO-542, TNX-355, SCH—C, and a combination thereof.

42. The method of claim 33, wherein the animal is a human.
